(19) United States
(12) Patent Application Publication
(10) Pub. No.: US 2023/0031483 A1
(43) Pub. Date: Feb. 2, 2023

(54) INDIVIDUALIZED VACCINES FOR CANCER TREATMENT AND PREVENTION

(71) Applicant: MAYO FOUNDATION FOR MEDICAL EDUCATION AND RESEARCH, Rochester, MN (US)

(72) Inventors: Asher Alban Akmal Chanan-Khan, Jacksonville, FL (US); Alak Manna, Jacksonville, FL (US); Aneel Paulus, Ocala, FL (US)

(21) Appl. No.: 17/788,993

(22) PCT Filed: Dec. 29, 2020
(86) PCT No.: PCT/US2020/067237

§ 371 Date: Jun. 24, 2022

Related U.S. Application Data
(60) Provisional application No. 62/955,684, filed on Dec. 31, 2019.

(51) Int. Cl. A61K 39/00

(52) U.S. Cl. CPC A61K 39/0011; A61K 2039/5152

(57) ABSTRACT

The present invention includes a method of preparing apoptotic bodies from a tumour and immunogenic compositions thereof. The method of preparation comprises: obtaining human tumour cells from a subject, inducing apoptosis of the human tumour cells with a drug or a physical treatment, and collecting apoptotic bodies from the apoptotic human tumour cells by centrifugation. The method comprises two centrifugation steps, low-speed at 50 g for 5 minutes to pellet cells, followed by high-speed centrifugation of the obtained supernatant at 3,000 g for 8 minutes to pellet apoptotic bodies. The purity of the apoptotic bodies (also referred to as immunogenic Tumor Apoptotic Bodies (TABi)) was determined by FACS to be 82.22%.

### CROSS-REFERENCE TO RELATED APPLICATIONS

This application claims priority to U.S. Provisional Application Ser. No. 62/955,684, filed Dec. 31, 2019, the entire contents of which are incorporated herein by reference.

### TECHNICAL FIELD OF THE INVENTION

The present invention relates in general to the field of Tumor Apoptotic Body Immunization (TABI), and more particularly, to compositions and methods for developing and using individualized vaccines for cancer treatment and prevention.

### STATEMENT OF FEDERALLY FUNDED RESEARCH

No federal/government funding was used to develop this invention.

### BACKGROUND OF THE INVENTION

Without limiting the scope of the invention, its background is described in connection with making and using tumor apoptotic bodies.

One such example is taught in U.S. Patent Publication No. 2018/0125956, filed by Fucikova, et al., entitled “Cryopreservation Of Apoptotic Cancer Cells For Use In Immunotherapy Against Cancer”. These applicants are said to teach a method for preparing a potent vaccine useful for immunotherapy by cryopreserving a population of cells undergoing immunogenic cell death, and using such cells to activate dendritic cells for use in immunotherapy. In one example, the method uses cryopreserving cancer cells undergoing cell death, which are used to prepare a pharmaceutical composition for immunotherapy against cancer.

Another example is taught in International Patent Publication WO99/58645, filed by Gregoire and Bartholeyns, entitled “New apoptotic bodies, monocyte derived cells containing the same, a process for their preparation and their uses as vaccines”. These applicants are said to teach apoptotic bodies derived from human tumor cells or cell lines recovered from a patient's tumor biopsy and induced to apoptosis, in which the apoptotic bodies have the following characteristics: they maintain plasma membrane integrity; they are vesicles above about 0.1 μm; they have intact mitochondria and cleaved nuclear DNA originating from the tumor cells; they present unmasked tumor antigens on their membranes; and they present specific tumor and MHC antigens from the patient.

However, other artisans have found that tumor apoptotic bodies inhibit immune responses. Xie, et al., in an article entitled, “Tumor Apoptotic Bodies Inhibit CTL Responses and Antitumor Immunity via Membrane-Bound Transforming Growth Factor-B1 Inducing CD8+ T-Cell Anergy and CD4⁺ Tr1 Cell Responses”, Cancer Res 2009; 69: (19). Oct. 1, 2009, found that tumor apoptotic bodies were tolerogenic and capable of suppressing antigen-stimulated CD8⁺ CTL responses and antitumor immunity via its induction of CD8⁺ T-cell anergy and type 1 regulatory CD4⁺ T-cell responses.

Thus, it is unclear from the art how to make tumor apoptotic bodies that may be used to stimulate the immune response. What are needed are new methods and compositions that overcome the known problems with the prior art.

### SUMMARY OF THE INVENTION

In one embodiment, the present invention includes a method of preparing apoptotic bodies from a tumor biopsy or specimen, comprising: obtaining human tumor cells from a subject; inducing apoptosis of the human tumor cells with a drug or a physical treatment; and collecting apoptotic bodies from the apoptotic human tumor cells by: centrifuging at a low speed to form a pellet and a low speed centrifugation supernatant; collecting the low speed centrifugation supernatant; and centrifuging the low speed centrifugation supernatant at a high speed to form a high speed centrifugation pellet and a high speed centrifugation supernatant; and isolating the high speed centrifugation pellet, wherein an immunogenic composition comprises drug-treated immunogenic Tumor Apoptotic Bodies (TABi) having a purity of at least 82 to 100 percent, and wherein the TABI are obtained in less than 2 hours. In one aspect, the step of centrifuging at low-speed is repeated prior to the high-speed centrifugation step. In another aspect, the step of inducing apoptosis is with more than one drug directed to that human tumor cell type. In another aspect, the method further comprises the step of obtaining the human tumor cells selecting the drug or physical treatment(s) to make subject-specific TABis, measuring cTL proliferation in vitro to the subject-specific TABi, and then providing the subject-specific TABIs to the subject depending on the cTL proliferation measured. In another aspect, the step of centrifuging at low-speed is at less than 100 g, or between 25 and 75 g. In another aspect, the step of centrifuging at high-speed is at greater than 1,500 g, or between 1,750 and 10,000 g. In another aspect, the method does not include a medium-speed centrifugation step, wherein the medium-speed centrifugation is between 100 and 1,500 g. In another aspect, the human tumor cells are from a liquid or a solid tumor. In another aspect, the human tumor cells are obtained from lung, breast, cervical, ovarian, esophageal, colon, rectum, neural, glioma, prostate, bladder, bone, pancreas, liver, ovary, testis, uterus, placenta, brain, cartilage, smooth muscle, striated muscle, fibrous tissue, blood vessel, lymph vessel, lymph node, adipose tissue, kidney, pituitary gland, parathyroid, thyroid, bronchial, adrenal, stomach, large intestine, small intestine, skin, adenomas, sarcomas, carcinomas, leukemias, lymphomas, multiple myeloma or blastomas, or a combination of any thereof. In another aspect, the TABI are ToPro-3 and Annexin-V positive, and following the isolation step comprise greater than 82% of total cells. In another aspect, the TABI are autologous, allogeneic, or xenogeneic. In another aspect, the drug used to treat the tumor cells is selected from an antimetabolite agent, alkylating or alkylating-like agent, intercalating agent, topoisomerase I or II inhibitor, antimitotic agent, kinase inhibitor, proteasome inhibitor, Bcl-2 inhibitors, chemotherapy agents, monoclonal antibodies, biologics, targeted therapeutics. Some specific examples of agents include butyrate derivatives, staurosporine, sulindac derivatives, inflammatory cytokines, 5-fluorouracil, capecitabine, gemcitabine, pemetrexed, methotrexate, pemetrexed, methotrexate, edatrexate, hydroxyurea, fludarabine, mercaptopurine, nitrogen mustards (mechlorethamine, cyclophosphamide, ifosfamide, melphalan and chlorambucil), aziridines (thiotepa), nitrosoureas (carmustine, lomustine, semustine), triazenes (dacarbazine and temozolomide) and platinum derivatives (cisplatin, oxaliplatin, carboplatin and satraplatin), doxorubicin, epirubicin, idarubicin, nemorubicin, mitoxantrone, etoposide, teniposide, paclitaxel, docetaxel, ixabepilone, vinblastine, vincristine, vindesine and vinorelbine, sorafenib, dasatinib, gefitinib, erlotinib, sunitinib, imatinib, nilotinib lapatinib, ibrutinib, acalabrutinib, bevacizumab (antibody to vascular endothelial growth factor), cetuximab, panitumumab, matuzumab, nimotuzumab (antibodies to epidermal growth factor receptor), trastuzumab, pertuzumab (antibodies to ErbB2), daratumumab (antibody to CD38), anti-CD20 inhibitors (rituximab, obinutuzumab, ofatumumab), topotecan, SN-38, CPT11, 9-nitrocamptothecin, or antineoplasic nucleoside analogs. In another aspect, the physical treatment for inducing apoptosis is by a physical method selected from: ionization, γ-irradiation, UV irradiation, heat shock, stress, serum deprivation, or a combination thereof. In another aspect, the TABI are lyophilized. In another aspect, the TABI are cryogenically stored in an aqueous solution comprising HEPES, NaCl, KCl, MgCl, vesicle free autologous serum and Trehalose, adjusted to pH 7.4.

In one embodiment, the present invention includes an immunogenic Tumor Apoptotic Body Immunization (TABI) composition made by a method comprising: obtaining human tumor cells from a subject; inducing apoptosis of the human tumor cells with a drug or a physical treatment; and collecting apoptotic bodies from the apoptotic human tumor cells by: centrifuging at a low-speed to form a pellet and a low speed centrifugation supernatant; collecting the low-speed centrifugation supernatant; and centrifuging the low speed centrifugation supernatant at a high speed to form a high-speed centrifugation pellet and a high-speed centrifugation supernatant; and isolating a pellet after the high-speed centrifugation, wherein the pellet comprises immunogenic Tumor Apoptotic Bodies (TABi) having a purity of at least 82 percent, and wherein the TABi are obtained in less than 2 hours. In one aspect, the step of centrifuging at low-speed is repeated prior to the high-speed centrifugation step. In another aspect, the step of inducing apoptosis is with more than one drug directed to that human tumor cell type. In another aspect, the step of centrifuging at low-speed is at less than 100 g, or between 25 and 75 g. In another aspect, the step of centrifuging at high-speed is at greater than 1,500 g, or between 1,750 and 10,000 g. In another aspect, the method does not include a medium-speed centrifugation step, wherein the medium-speed centrifugation is between 100 and 1,500 g. In another aspect, the human tumor cells are from a liquid or a solid tumor. In another aspect, the human tumor cells are obtained from lung, breast, cervical, ovarian, esophageal, colon, rectum, neural, glioma, prostate, bladder, bone, pancreas, liver, ovary, testis, uterus, placenta, brain, cartilage, smooth muscle, striated muscle, fibrous tissue, blood vessel, lymph vessel, lymph node, adipose tissue, kidney, pituitary gland, parathyroid, thyroid, bronchial, adrenal, stomach, large intestine, small intestine, skin, adenomas, sarcomas, carcinomas, leukemias, lymphomas, multiple myeloma or blastomas, or a combination of any thereof. In another aspect, the TABi are ToPro-3 and Annexin-V positive, and following the isolation step comprise greater than 82% of total cells. In another aspect, the TABi or the TABI are autologous, allogeneic, or xenogeneic. In another aspect, the agents used to treat the tumor cells is selected from an antimetabolite agent, alkylating or alkylating-like agent, intercalating agent, topoisomerase I or II inhibitor, antimitotic agent, kinase inhibitor, proteasome inhibitor, Bcl-2 inhibitors, chemotherapy agents, monoclonal antibodies, biologics, targeted therapeutics, e.g., butyrate derivatives, staurosporine, sulindac derivatives, inflammatory cytokines, 5-fluorouracil, capecitabine, gemcitabine, pemetrexed, methotrexate, pemetrexed, methotrexate, edatrexate, hydroxyurea, fludarabine, mercaptopurine, nitrogen mustards (mechlorethamine, cyclophosphamide, ifosfamide, melphalan and chlorambucil), aziridines (thiotepa), nitrosoureas (carmustine, lomustine, semustine), triazenes (dacarbazine and temozolomide) and platinum derivatives (cisplatin, oxaliplatin, carboplatin and satraplatin), doxorubicin, epirubicin, idarubicin, nemorubicin, mitoxantrone, etoposide, teniposide, paclitaxel, docetaxel, ixabepilone, vinblastine, vincristine, vindesine and vinorelbine, sorafenib, dasatinib, gefitinib, erlotinib, sunitinib, imatinib, nilotinib lapatinib, ibrutinib, acalabrutinib, bevacizumab (antibody to vascular endothelial growth factor), cetuximab, panitumumab, matuzumab, nimotuzumab (antibodies to epidermal growth factor receptor), trastuzumab, pertuzumab (antibodies to ErbB2), daratumumab (antibody to CD38), anti-CD20 inhibitors (rituximab, obinutuzumab, ofatumumab), topotecan, SN-38, CPT11, 9-nitrocamptothecin, or antineoplasic nucleoside analogs. In another aspect, the physical treatment for inducing apoptosis is by a physical method selected from: ionization, γ-irradiation, UV irradiation, heat shock, stress, serum deprivation, or a combination thereof. In another aspect, the TABi or the TABI are lyophilized. In another aspect, the TABi are cryogenically stored in an aqueous solution comprising HEPES, NaCl, KCl, MgCl, vesicle free autologous serum and Trehalose, adjusted to pH 7.4. In another aspect, the method further comprises the step of obtaining the human tumor cells selecting the drug or physical treatment(s) to make subject-specific TABis, measuring cTL proliferation in vitro to the subject-specific TABi, and then providing the subject-specific TABIs to the subject depending on the cTL proliferation measured.

A method of preparing apoptotic bodies from a tumor biopsy, comprising: obtaining human tumor cells from a subject; inducing apoptosis of the human tumor cells with a drug or a physical treatment; and collecting apoptotic bodies from the apoptotic human tumor cells by: centrifuging at a low speed of less than or equal to 100 g to form a pellet and a low speed centrifugation supernatant; collecting the low speed centrifugation supernatant; and centrifuging the low speed centrifugation supernatant at a high speed of greater than 1,500 g to form a high speed centrifugation pellet and a high speed centrifugation supernatant; and isolating the high speed centrifugation pellet, wherein an immunogenic composition comprises drug-treated immunogenic Tumor Apoptotic Bodies (TABi) having a purity of at least 82 to 100 percent, and wherein the TABi are obtained in less than 2 hours.

### BRIEF DESCRIPTION OF THE DRAWINGS

For a more complete understanding of the features and advantages of the present invention, reference is now made to the detailed description of the invention along with the accompanying figures and in which:

FIG. 1 shows the basic steps for preparing the immunogenic tumor apoptotic bodies (TABi) of the present invention.

FIG. 2 shows three flowcharts that compare the present invention (middle flowchart) from methods of the prior art (left and right flowcharts).

FIG. 3 shows fluorescent activated cell sorting (FACS) results that compare the results using the prior art methods (left and right), with the present invention in the middle.

FIG. 4 shows FACS results from using the method of the present invention to select immunogenic TABi, post sort purity check using flow cytometer.

FIGS. 5A to 5C show FACS results from TABis derived under various treatment conditions.

FIGS. 6A to 6C show the generation and immunogenic properties of tumor apoptotic bodies (TABi) of the present invention.

FIG. 7 shows the quantification of CTL proliferation after pulsing with patient-autologous TABis of the present invention.

FIG. 8 shows that TABi-primed CD8+ T-cells from CLL patients induce robust specific-lysis of autologous CLL tumor cells.

FIG. 9 shows the gating strategies for characterization of DNA contents of TABis of the present invention.

FIG. 10 shows the gating strategies for characterization of mitochondrial contents of TABis of the present invention.

FIGS. 11A to 11E shows the content of TABis: Intracellular contents of TABis were imaged using confocal microscopy and quantified using flow cytometery.

FIG. 12 shows the co-culture of PBMCs from CLL patients with autologous TABi's generated with various therapeutic agents followed by measurement of patient-autologous cTL proliferation.

FIG. 13 shows the quantification of cTL proliferation after pulsing with patient-autologous TABis.

FIG. 14 shows TABi-primed CD8+ T-cells from CLL patients induce robust specific-lysis of autologous CLL tumor cells.

FIGS. 15A and 15B shows that TABI's stimulate patient tumor-specific cytolytic T-cell activity.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “apoptotic bodies” refers to cell fragmentation bodies that include an intact membrane, mitochondria, and fragmented nuclear DNA from tumor cells induced to apoptosis by either a drug/chemical treatment and/or a physical insult or treatment (e.g., exposure to UV light or X-rays). Apoptotic cells are distinguishable from necrotic cells according to characteristic morphological and biochemical features known in the art. Programmed cell death or “apoptosis” is characterized by one or more of the following: chromatin aggregation followed by DNA fragmentation (a marker of an apoptotic process) after activation of endonucleases resulting in DNA fragments of, typically, 180 basepairs, cell shrinkage, reorganization of the cell nucleus, cell membrane and cell metabolism, active membrane blebbing, and ultimate fragmentation of the cell into membrane-enclosed vesicles, known as apoptotic bodies. The nuclear events leading to apoptosis begin with collapse of the chromatin against the nuclear periphery and into one or a few large clumps within the nucleus. The cellular events include cytoplasmic condensation and partition of the cytoplasm and nucleus into membrane bound-vesicles (apoptotic bodies), which contain ribosomes, intact mitochondria and nuclear material which are surrounded by an intact cellular membrane (a specific marker of apoptotic process when compared with necrosis, the other non physiological cell death process).

As used herein, the term “cancer” refers a metastatic and/or a non-metastatic cancer, and includes primary and secondary cancers.

As used herein, the terms “biological sample” or “sample” refer to materials obtained from, or derived from, a subject or patient and can include sections of tissues such as blood, a biopsy, a solid tumor, a liquid tumor, a tumor, or even cancer cells. Biological samples can include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc.

A “biopsy” refers to the process of removing a tissue sample for growing cells, diagnostic or prognostic evaluation, and to the tissue specimen itself. Any biopsy technique known in the art can be applied to the diagnostic and prognostic methods disclosed herein. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), the size and type of a tumor (i.e., solid or suspended (i.e., blood or ascites)), among other factors. Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

As used herein, the term “liquid biopsy” refers to a biological sample obtained from a body fluid such as blood, urine, cerebrospinal fluid (CFS), aqueous or vitreous or abdominal cavity fluid, lymph node fluid, bladder fluid, milk duct fluid, sputum, gastric fluid, bile duct fluid, sinus fluid, and combinations thereof.

As used herein, the terms “treating” or “treatment”, as are well understood in the art, refer to obtaining beneficial or desired results, including clinical results, when treating a subject with cancer or other disease. Using the present invention, beneficial or desired clinical results can include, e.g., amelioration or alleviation of one or more symptoms or conditions, diminishment of extent of cancer disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. Thus, “treating” and “treatment” can also include prolonging survival as compared to expected survival when not receiving treatment. As used herein, “treating” and “treatment” using the immunogenic TABis of the present invention also include a prophylactic treatment. For example, a subject with early stage cancer can be treated to prevent progression or metastases, or alternatively a subject in remission can be treated with a compound or composition described herein to prevent recurrence. Typically, treatment methods include administering to a subject a therapeutically effective amount of the TABis of the present invention, whether as a single administration, or more commonly, as a series of immunizations. For example, a tumor apoptotic body immunization (TABI) of the present invention may be administered at least once a week. Alternatively, the TABI of the present invention may be administered to the subject from about one time every two to three weeks, or about one time per week for a given treatment regimen. The course or length of treatment depends on a variety of factors, such as the severity of the disease, the age of the patient, the concentration, the activity of the TABI of the present invention, and/or combinations thereof. It will also be appreciated that the effective dosage of the TABI of the present invention used for treatment or prophylaxis may increase or decrease over the course of a particular treatment or prophylaxis regimen. Changes in dosage of the TABI of the present invention may result and become apparent by standard diagnostic assays or imaging known in the art. In some instances, chronic administration may be required. For example, the TABIs of the present invention are administered to the subject in an amount and duration sufficient to treat the patient.

As used herein, the following abbreviations are used, a TABI is a Tumor Apoptotic Body Immunization, which is the process of immunization. By contrast, a TABi is the Tumor Apoptotic Bodies that are used in the immunization, and TABis refer to the physical entity, i.e., an apoptotic body isolated from the cell.

The present inventors have discovered that apoptotic bodies of cancer cells can induce an immune mediated anti-tumor response. This observation was used to develop personalized vaccines for patients with cancers. Using a unique strategy for generation of these apoptotic bodies, the present inventors generated a spectrum of immunogenic tumor apoptotic bodies (TABi) from the same tumor type, in which each of these TABis have a unique and differential effect on the antitumor immune response. The TABis can be used in a tumor apoptotic body Immunization (TABI). Alone or together, the methods and composition taught herein allowed for generating a TABI with sufficient TABi's for preparing immunizations or vaccinations, with a high therapeutic effect, that when stored long-term had an equivalent or only slightly reduced effectiveness when compared to fresh vaccine.

Generation of apoptotic bodies. The unique aspect of generating immune-activating TABi's encompasses their utilization in anticancer therapy. Primary tumor cells from patients were removed and subject to cytolysis ex vivo, using various standard-of-care therapeutic agents. It was found that each therapeutic agent resulted in the generation of TABi's that are subjectively/physically different; translating into a variable immunologic potential of each type of TABi, and for the combination of TABi's in each vaccine. This vaccine approach can be used with a panel of anticancer therapeutics. The specific anticancer therapeutic(s) will vary and will be dependent upon the tumor type, as will be known to the skilled artisan based on the, then-current, standard of care for the specific cancer type. Using the methods of the present invention, it was possible to generate a sufficient amount of patient-derived TABi's from, and for, vaccination of the subject.

Selection of anti-cancer therapeutics for generation of TABi. TABis are generated by a unique panel of anticancer drugs that will be specific to a patient as well as specific to the type of cancer. This unique strategy generate vaccines for TABi that is based on the therapeutic agents used, which will be determined through the following process: (a) disease specific anticancer drugs: for each tumor type a panel of up to 5 therapeutics (or physical treatments) are typically used in the clinic to treat that cancer type (it is possible to use more than 5, but generally up to 5 will be commonly used, which may include combinations of drugs and physical treatments such as UV, X-ray, heat shock, etc., as will be described herein). Selection of these 1-5 therapeutic agent(s)/treatment(s) is determined based upon the TABi's generated with the highest capability of immunogenic response. Primary tumor cells from the patient will be exposed to IC⁵⁰⁻⁷⁰ concentrations of each of these agents (ex vivo) independently, which would result in generation TABis, that would be stored. (b) Immunologic potential of specific TABis, in which each of the individual TABis generated (alone or in combination) are used to stimulate T-cells (ex vivo) for assessment of their immune stimulatory potential. TABis with the highest immunogenicity (as determined by T-cell proliferation assay) are pooled together for that specific patient and used as a TABI. (c) Immunization of the patient using a single dose, but more commonly, an immunization plan in which multiple immunizations are applied over a course of time.

Storage of TABi. The present inventors have found that TABi's must be stored in a specific manner to maintain their immunogenic potential. Also, the ratio of each TABi generated within the TABI vaccine is unique and specifically designed for each tumor type and for each individual patient—making each TABI vaccine highly individualized.

Process of generation of TABI immunization/vaccine: TABI immunization will be generated for each specific patient after removal of the cancer tissue from the individual. Each TABI vaccine will be developed as follows: (1) Tumor cells are isolated from the subject, and treated with the panel of up to 5 therapeutics or physical treatments, leading to apoptosis and TABi formation. While not preferred, it is also possible to use flow cytometry (or equivalent methodology) to make single tumor cell preparations. These single tumor cells are cultured in vitro and treated to generated TABis with the panel of up to 5 therapeutics or physical treatments. (2) The TABis are isolated using the low-speed/high-speed centrifugation method of the present invention, to provide a significant amount of isolated TABis, within 2 hours. (3) Concurrently, ex vivo cell expansion via a patient-derived tumor xenograft (PDx) model to expand the tumor tissue volume for adequate material to generate the TABI vaccine. (4) The TABis are stored with the novel composition of the present invention, providing long-term storage of an effective TABI immunization. The TABi can be formulated into the TABI using compositions and methods known to the artisan skilled in preparing immunizations.

Clinical utilization of TABI. Typically, the present invention will be used with or after a cancer treatment, however, it is also possible to use the TABIs as a single or frontline therapeutic agent. TABI immunization can be used as an adjuvant to systemic induction treatment in an effort to (1) delay relapse, (2) eradicate minimal residual disease (MRD), (3) use as an immunotherapy to re-induce remission in patients with relapse, and/or (4) ex vivo priming and expansion of tumor directed T cells or other immune cells.

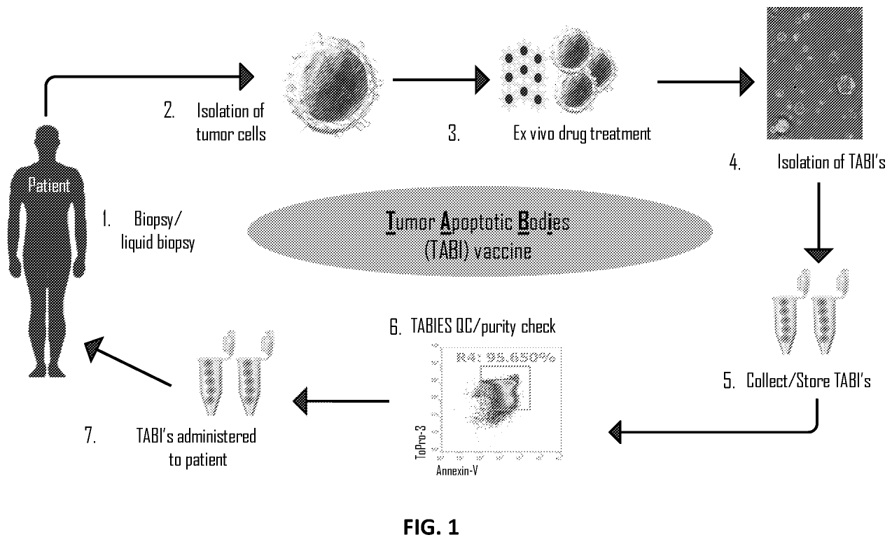

FIG. 1 shows the basic steps for preparing the immunogenic tumor apoptotic bodies (TABi) of the present invention, using leukemia as an example. The skilled artisan will recognize that solid tumor biopsies can be substituted for the leukemia. In step 1, a biological sample, such as blood, liquid biopsy, lymph node, etc., is collected from a patient with a cancer, e.g., a chronic lymphocytic leukemia (CLL). The skilled artisan will recognize that many different sources of cancer cells can be used with the present invention. In step 2, tumor cells are isolated. For example, CD19+CD5+ CLL cells can be isolated from the blood sample, and in particular the CD19+CD5+ CLL cells are separated from the red blood cells and from other cells, e.g., white blood cells, by lysing the red blood and non-CD19+CD5+ CLL cells, panning or adhering the CD19+CD5+ CLL cells, or other method of isolation. In step 3, the tumor cells are treated ex vivo with a drug or other treatment. For example, CD19+CD5+ CLL cells are treated ex vivo with a drug or physical method that triggers the formation of membrane blebs, leading to the formation of immunogenic tumor apoptotic bodies (TABi). In step 4, the TABis are isolated using the novel low-speed centrifugation method of the present invention. In step 5, the purity of the TABis is confirmed, and in step 6, the TABis are prepared into the TABI immunization used to treat, in which example, the same patient.

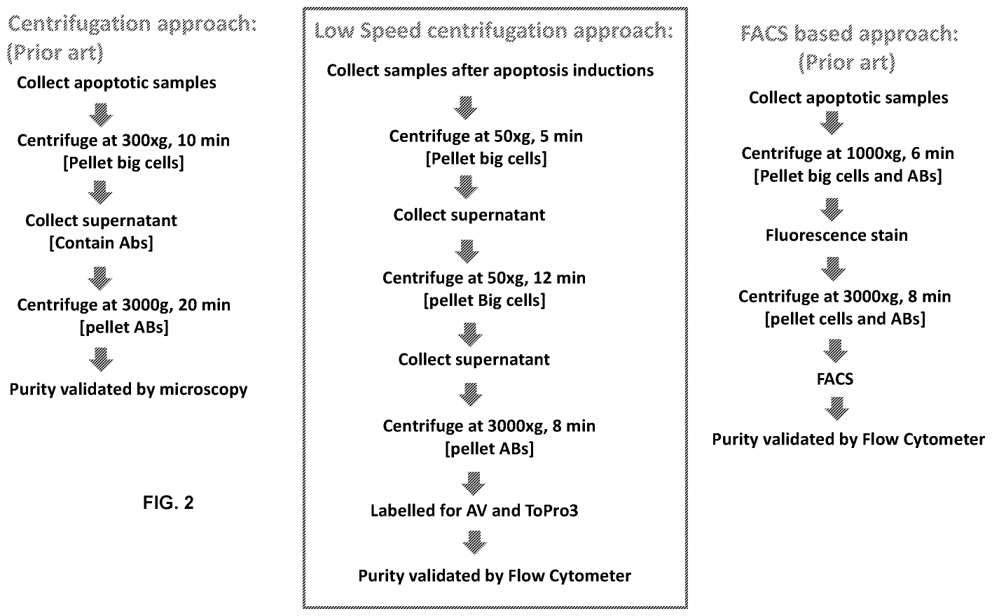

FIG. 2 shows three flowcharts that compare the present invention (middle flowchart) from methods of the prior art (left and right flowcharts). The traditional methods for isolating tumor apoptotic bodies are shown the left and right. In the standard centrifugation approach, apoptotic bodies are formed and subjected to a medium-speed centrifugation, typically at 300xg for 10 minutes to pellet the larger cells. The medium-speed centrifugation supernatant is collected and subjected to a high-speed centrifugation, typically at 3,000xg for 20 minutes, with the purity checked by microscopy/flow cytometry. In the fluorescence sorting method of the prior art, the apoptotic bodies are collected and centrifuged at a high-speed of 1,000xg for 6 minutes to pellet big cells and apoptotic bodies, which are then stained. The stained cells and apoptotic bodies are then centrifuged at a higher speed of 3,000xg to eliminate the cells, and the supernatant is subjected to fluorescence activated cell sorting (FACS) for about 4-6 hours to obtain sufficient apoptotic bodies for testing, and the resulting apoptotic bodies are verified by FACS. By contrast, the present invention obtains the apoptotic bodies and first subjects them to at least one low-speed centrifugation step of about 50xg for about 5 minutes. This low-speed supernatant is collected and then subjected to a high-speed centrifugation step of about 3,000xg for 8 minutes, leading to a significantly higher yield. To test purity and percentage of the apoptotic bodies (versus non-apoptotic bodies), a small sample of the apoptotic bodies is labeled with markers, such as Annexin-V and ToPro3, to validate the purity by flow cytometry.

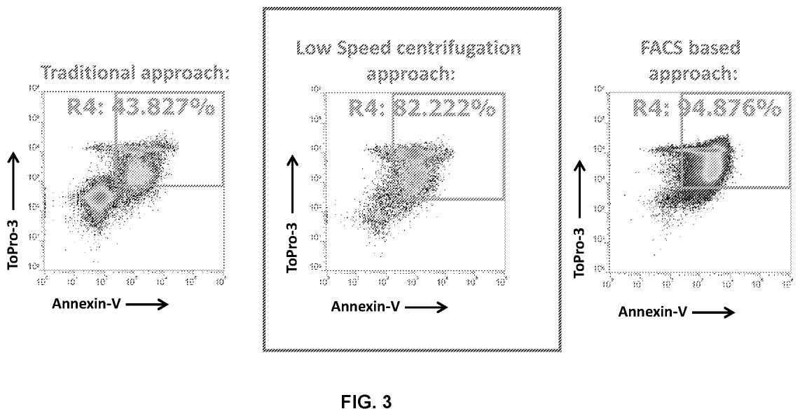

FIG. 3 shows fluorescent activated cell sorting (FACS) results that compare the results using the prior art methods (left and right), with the present invention in the middle. Method to select low-speed centrifugation approach. Quantity, purity, time and cost efficiency of TABis was considered in selection of the low-speed centrifugation approach. Using this method, the purity (˜82%) and quantity of apoptotic cells (Annexin-V+/ToPro-3+) was optimal and achieved within 1.5 hours. Contrastingly, traditional centrifugation yielded lower purity (˜43%). While FACS-based approach yielded higher purity (94%), this method took ˜12-14 hours. All data were analyzed using FCS Express 6 (DeNovo Software).

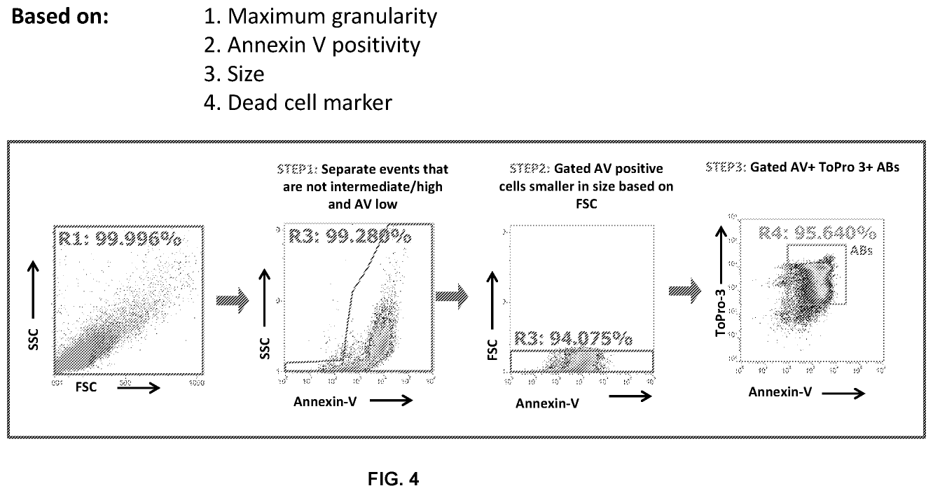

FIG. 4 shows FACS results from using the method of the present invention to select immunogenic TABi, post sort purity check using flow cytometer. Method to select immunogenic TABi-post sort purity check using flow cytometer. After treatment with an anti-CD38 monoclonal antibody (Daratumumab) (30 μg/mL, 18h), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide (Venetoclax) (30 μM, 18h) and Ultraviolet beam (254 nm, 17 min, followed by incubation for 18h) CD19+ sorted CLL cells were stained with Annexin V and/or TO-PRO-3 in Annexin V binding buffer (1×) for 10 min at room temperature and immediately placed on ice before analysis on a Attune NxT flow cytometer (invitrogen). Post sort purity of TABis were determined using following gating strategies (Annexin V positive with low forward scatter (FSC) confirmed with Annexin V+/TO-PRO-3+). Unstained samples were used as a control. All data were analyzed using FCS Express 6 (DeNovo Software).

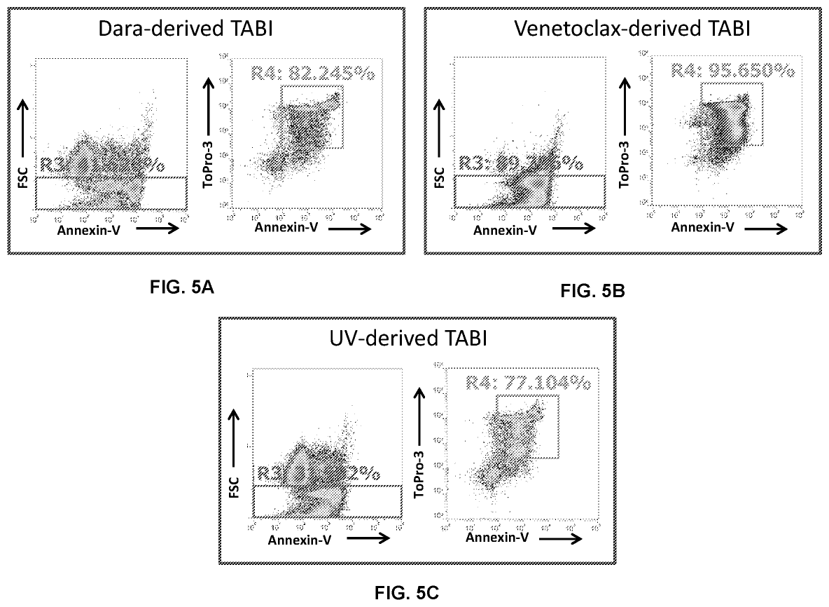

FIGS. 5A to 5C show FACS results from TABis derived under various treatment conditions. TABis derived under various treatment conditions. CD19+/CD5+ CLL cells (n=3 patients) were treated with: FIG. 5A supratherapeutic concentrations of anti-CD38 therapeutic monoclonal antibody daratumumab (Dara), FIG. 5B Bcl-2 inhibitor venetoclax (Ven), or FIG. 5C short wave UV light for 24 hours. Cells were subjected to flow cytometry and gated based on size (FSC) and apoptotic marker (Annexin-V) as shown in Gate R3. These gated cells were then further examined for TABi purity as shown in Gate R4.

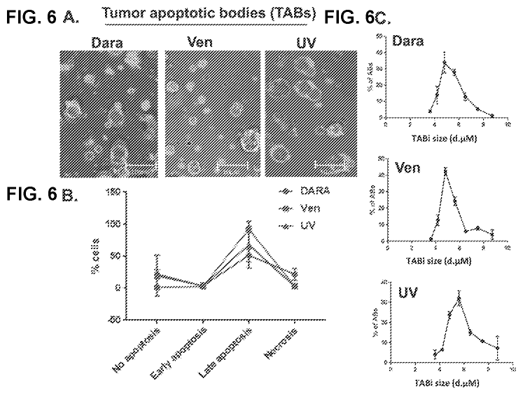

FIGS. 6A to 6C show the generation and immunogenic properties of tumor apoptotic bodies (TABi). FIG. 6A. TABis were harvested from CD19+/CD5+ CLL cells (n=3 patients) that were treated with supratherapeutic concentrations of anti-CD38 therapeutic monoclonal antibody daratumumab (Dara), Bcl-2 inhibitor venetoclax (Ven) or short wave UV light for 18h. TABis were stained with DAPI (blue) and Annexin-V (green, marker of apoptosis) and imaged by confocal microscopy. FIG. 6B. Separately, TABis were stained with Annexin-V and propidium iodide and subjected to flow cytometry to delineate which stages of apoptosis the TABis were experiencing over a 24 hr period. FIG. 6C. TABis size was determined by spectrophotometry, which showed that sizes ranged from 3.6 μm-8.7 μm and were to some extent dependent on the drug used to generate the TABis.

TABLE 1
Quantified Data from FIG. 6B and 6C

| Size distribution (d · μM) | DARA | VEN | UV |
|---|---|---|---|
| 3.6 | 4.00 ± 0.4 | 1.47 ± 0.75 | 4.07 ± 1.53 |
| 4.18 | 13.96 ± 3.74 | 12.9 ± 2.3 | 6.5 ± 0.3 |
| 4.8 | 33.95 ± 4.55 | 42.50 ± 1.6 | 23.75 ± 1.2 |
| 5.6 | 27.85 ± 1.35 | 24.35 ± 1.85 | 32.05 ± 2.6 |
| 6.5 | 13.00 ± 1.6 | 6.05 ± 0.45 | 15.20 ± 1.0 |
| 7.5 | 2.50 ± 0.1 | 7.9 ± 0.80 | 10.80 ± 0.2 |
| 8.7 | 1.25 ± 0.14 | 4.15 ± 2.05 | 7.30 ± 4.2 |

TABLE 2

| Stages of Apoptosis | DARA | VEN | UV |
|---|---|---|---|
| No apoptosis | 22.33 ± 3.95 | 1.25 ± 0.61 | 19.79 ± 18.59 |
| Early | 3.8 ± 1.42 | 2.6 ± 0.68 | 4.3 ± 2.31 |
| Late | 51.73 ± 5.73 | 92.47 ± 2.60 | 68.31 ± 21.05 |
| Necrosis | 22.16 ± 5.31 | 3.66 ± 2.48 | 3.07 ± 1.47 |

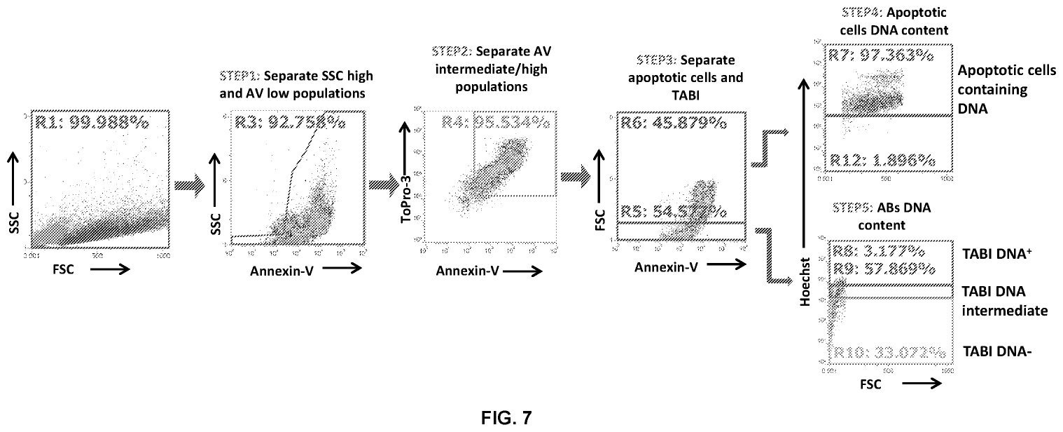

FIG. 7 shows another method of the present invention, in which the quantification of CTL proliferation after pulsing with patient-autologous TABis (T-cells derived from CLL patients, pulsed with autologous TABi from FIGS. 6A-6C) of the present invention. Quantification of CTL proliferation after pulsing with patient-autologous TABis (from Tables 1 and 2). CD8+ cytotoxic T-cells (cTLs) were isolated from CLL patients (n=3) and labeled with CFSE, followed by coculture with TABis generated from the autologous CLL cells (generated under similar conditions as in panel A). Incubation of cTLs with live TABis using Dara vs. Ven vs. UV induced robust proliferation of cTL in the order of 48.9%, 60.12% and 38.04%, respectively, compared to control (CFSE+ cTLs incubated with patient autologous DMSO-treated CLL cells). TABIs were stored under various conditions (shown in table) and freezing media in −80 F and thawed them 1 week later to test for their ability to induce a cTL immunogenic response. It was found that compared to commercial media (data on file), TABis stored in a TAB-stor (see Table 4), which when thawed and incubated with patient autologous CFSE+ cTLs, resulted in a similar level of cTL proliferation as compared to when fresh TABis were used in real-time. Table 3 includes the proliferation data.

TABLE 3
% CD8+ T-cell proliferation data for FIG. 7

| | Fresh TABis | TABstor TABis | Media only |
|---|---|---|---|
| Dara-TABis | 45.60 ± 2.80 | 28.01 ± 0.19 | 2.68 ± 0.18 |
| Ven-TABis | 59.61 ± 4.83 | 45.35 ± 4.25 | 2.00 ± 0.18 |
| UV-TABis | 39.13 ± 1.18 | 29.54 ± 0.01 | 1.88 ± 0.34 |

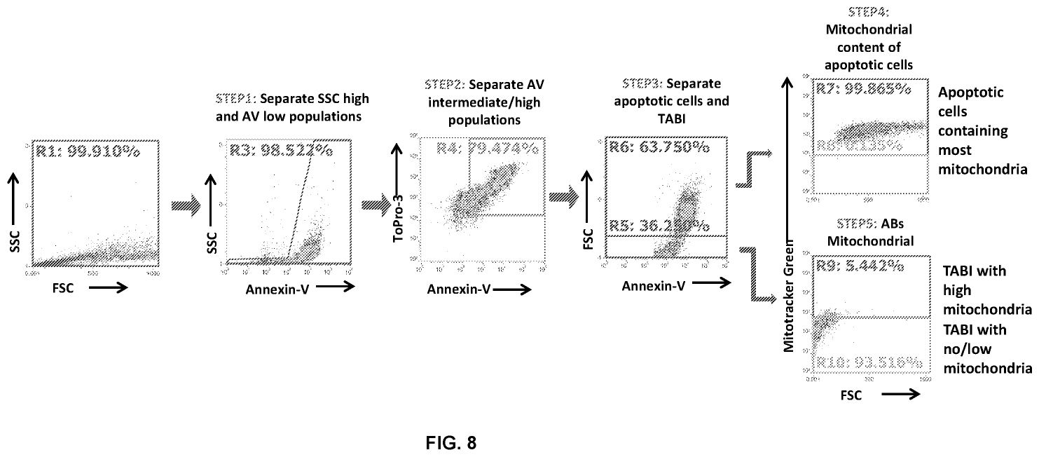

FIG. 8 shows another method of the present invention, in which TABi-primed CD8+ T-cells from CLL patients induce robust specific-lysis of autologous CLL tumor cells. TABi-primed CD8+ T-cells from CLL patients induce robust specific-lysis of autologous CLL tumor cells. Calcein AM labeled CD19+ B-cells from patients were isolated by MACs separation. Similarly CD8+ T-cells from the same patients were isolated and exposed to patient-autoglous TABis generated using various drugs (Daratumumab, D; venetoclax, V or UV light) for 72 hours. Hereafter, TABi-primed CD8+ T-cells were cocultured with Calcein-AM labeled autologous CLL cells for 6 hours and specific lysis was calculated. Non-TABi or drug containing media was used as a control. Effector (T-cell) to target (CLL cell) ratio was 1:1. Specific lysis was calculated using the formula:

$$Specific lysis = 100 \times \frac{Experimental release ( RFU ) - Spontaneous release ( RFU )}{Maximal release ( RFU ) - Spontaneous release ( RFU )} .$$

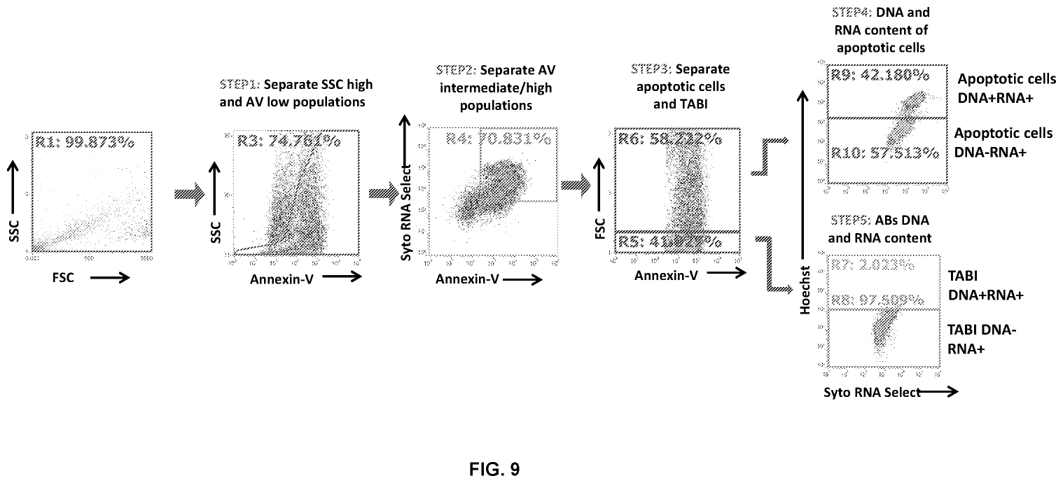

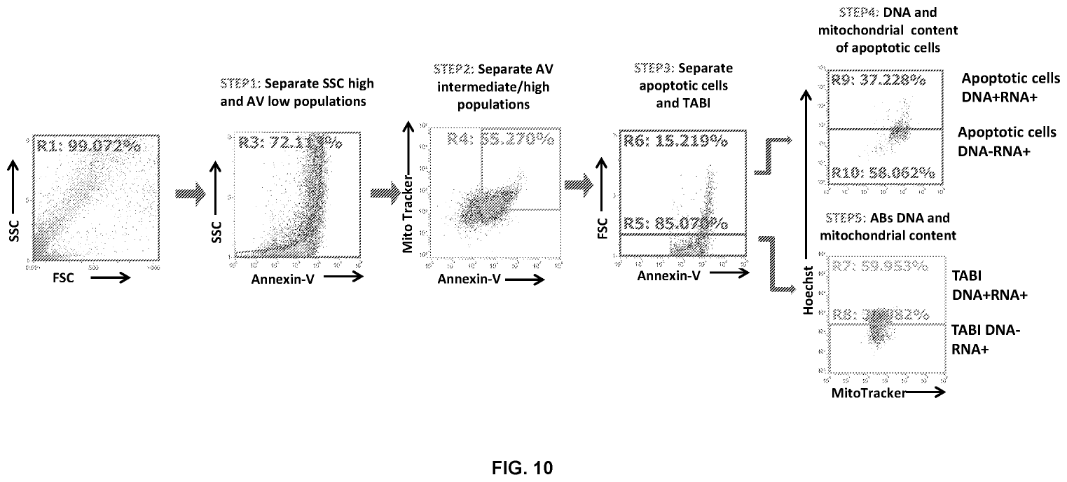

FIG. 9 shows another method of the present invention, in which the gating strategies for characterization of DNA contents of TABis of the present invention. FIG. 10 shows another method of the present invention, in which he gating strategies for characterization of mitochondrial contents of TABis of the present invention.

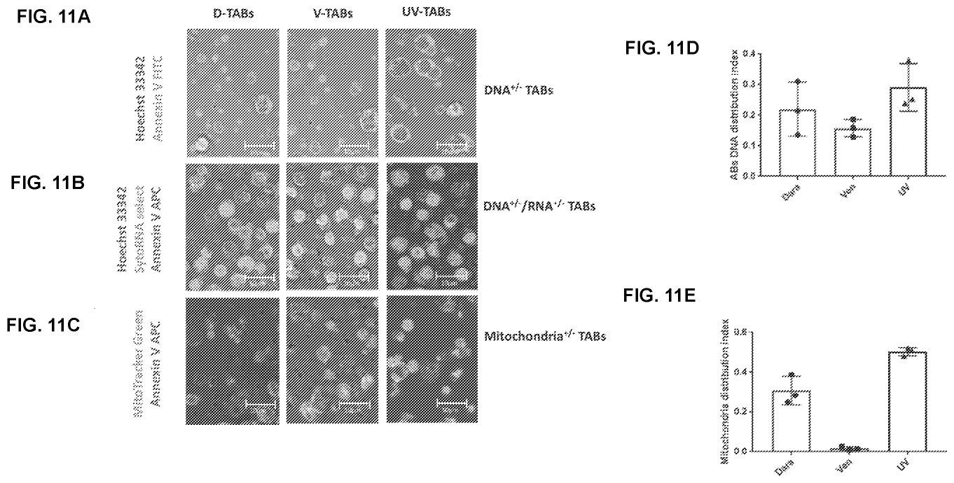

FIGS. 11A to 11E shows the content of TABis of the present invention: Intracellular contents of TABi's were imaged using confocal microscopy and quantified using flow cytometery. For confocal imaging, CD19+ sorted cells from CLL patients were pre-stained with (FIG. 11A) Hoechst 33342 (for DNA content); (FIG. 11B) Hoechst 33342 and SytoRNA select (for DNA and RNA content) and (FIG. 11C) MitoTracker Green (for mitochondrial content); followed by treatment with Daratumumab (D-TABis, 30 μg/mL, 18h), Venetoclax (V-TABis, 30 μM, 18h) or Ultraviolet beam (UV-TABis, 254 nm, 17 min, followed by incubation for 18h) to promote apoptotic cell disassembly. The cells were then stained with (FIG. 11A) Annexin V-FITC, and (FIGS. 11B and C) Annexin V-APC respectively. TABis were imaged using 63× magnification and data are representative of at least three independent experiments.

For flow cytometric analysis, CD19+ sorted CLL cells were stained with a combination of Hoechst 33342/Annexin V-FITC/TO-PRO-3 (DNA content) MitoTracker Green/Annexin V-V450/TO-PRO-3 (mitochondrial content), Hoechst 33342/MitoTracker Green/AnnexinV-APC (DNA and mitochondrial content), or Hoechst 33342/SYTO RNAselect green/Annexin V-APC (DNA and RNA content). Cells were stained with Hoechst 33342 (5 μg/ml), MitoTracker Green (100 nM) and/or SYTO RNAselect green (500 nM) prior to induction of apoptosis. Cells were stained with Annexin V and/or TO-PRO-3 in Annexin V binding buffer (1×) for 10 minutes at room temperature and immediately placed on ice before analysis on an Attune NxT flow cytometer (invitrogen). Unstained samples were used as a control. All data were analyzed using FCS Express 6 (DeNovo Software). Distribution of intracellular content of TABis varies with the mechanism of apoptotic cell disassembly using the various drugs. The distribution of DNA and mitochondria into TABis quantified based on (FIGS. 11D and 11E) TABis DNA distribution index (FIG. 11D) and TABi mitochondria distribution index (FIG. 11E), respectively (n=3). Data are representative of at least three independent experiments. DNA distribution index=DNA+ TABis/DNA− TABis; Mitochondria distribution index=mitochondria+ TABis/mitochondria− TABis.

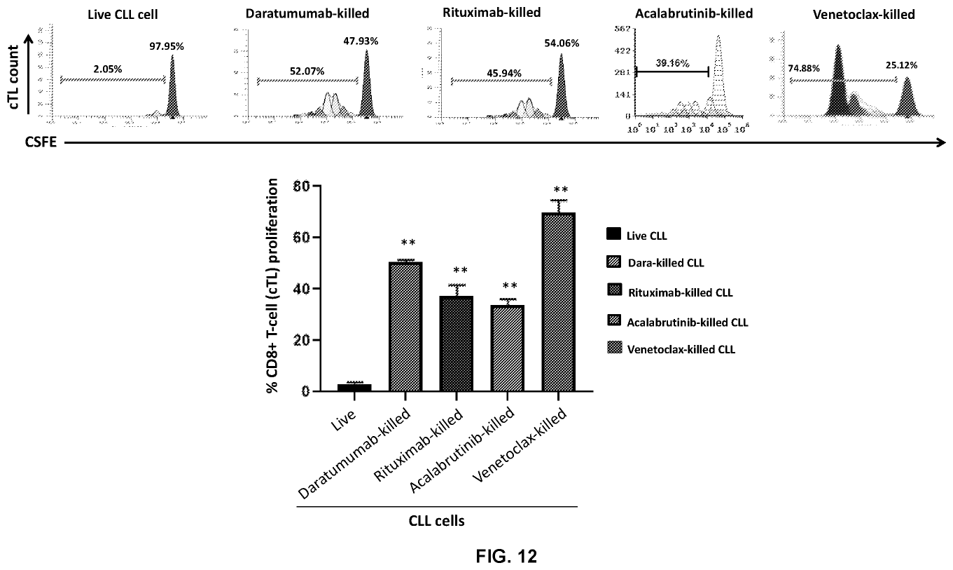

FIG. 12 shows that immunogenic tumor apoptotic bodies (TABis) generated with various anti-cancer agents induce robust cytotoxic CD8+ T cell proliferation. TABis were generated by exposing CD19+ sorted cells from CLL patients (n=3) with daratumumab (30 μg/mL), rituximab (30 μg/mL), acalabrutinib (30 μM) or venetoclax (30 μM, 18h); and were subsequently isolated using low speed centrifugation. In parallel, CD8+ T cells (cTL) were isolated and stained with CellTrace™ CFSE (5 μM, 20 min) and reintroduced back into their patient-autologous PBMCs mixture followed by co-culture with TABis for 72h at 37° C. and 5% CO₂. Cell were washed with antibody binding buffer and stained with CD4 and CD8. The proliferation of CD8 gated cells was determined using Attune NxT flow cytometer (Invitrogen). Data are representative of at least three independent experiments. **, p<0.001. All drugs were obtained from commercial or clinical sources.

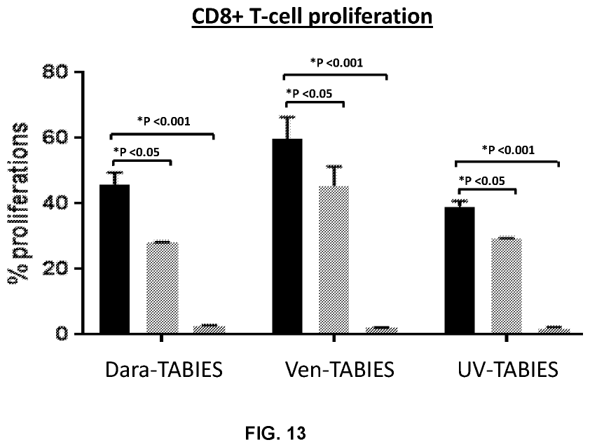

FIG. 13 shows the quantification of cTL proliferation after pulsing with patient-autologous TABis (from Slide 6D, E). CD8+ cytotoxic T-cells (cTLs) were isolated from CLL patients (n=3) and labeled with CFSE, followed by coculture with TABis generated from the autologous CLL cells (generated under similar conditions as in panel A). Incubation of cTLs with live TABis using Dara vs. Ven vs. UV induced robust proliferation of cTL in the order of 48.9%, 60.12% and 38.04%, respectively, compared to control (CFSE+ cTLs incubated with patient autologous DMSO-treated CLL cells). TABis stored under various conditions (shown in Table 6) and freezing media in −80° F. and thawed them 1 week later to test for their ability to induce a cTL immunogenic response. It was found that compared to commercial media (data on file), TABis stored in TAB-stor, which when thawed and incubated with patient autologous CFSE+ cTLs, resulted in a similar level of cTL proliferation as compared to when fresh TABis were used in real-time.

TABLE 5
% CD8+ T-cell proliferation in FIG. 13

| | Fresh TABis | TABstor TABis | Media only |
|---|---|---|---|
| Dara-TABis | 45.60 ± 2.80 | 28.01 ± 0.19 | 2.68 ± 0.18 |
| Ven-TABis | 59.61 ± 4.83 | 45.35 ± 4.25 | 2.00 ± 0.18 |
| UV-TABis | 39.13 ± 1.18 | 29.54 ± 0.01 | 1.88 ± 0.34 |

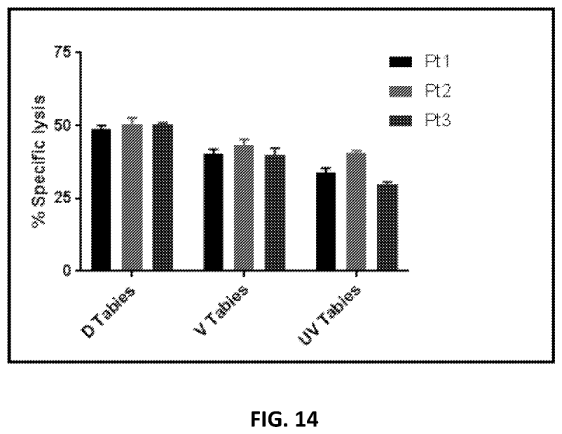

FIG. 14 shows TABi-primed CD8+ T-cells from CLL patients induce robust specific-lysis of autologous CLL tumor cells. Calcein AM labeled CD19+B-cells from patients were isolated by MACs separation. Similarly CD8+ T-cells from the same patients were isolated and exposed to patient-autoglous TABis generated using various drugs (Daratumumab, D; venetoclax, V or UV light) for 72 hours. Hereafter, TABi-primed CD8+ T-cells were cocultured with Calcein-AM labeled autologous CLL cells for 6 hours and specific lysis was calculated. Non-TABi or drug containing media was used as a control. Effector (T-cell) to target (CLL cell) ratio was 1:1. Specific lysis was calculated using the formula:

$$Specific lysis = 100 \times \frac{Experimental release ( RFU ) - Spontaneous release ( RFU )}{Maximal release ( RFU ) - Spontaneous release ( RFU )} .$$

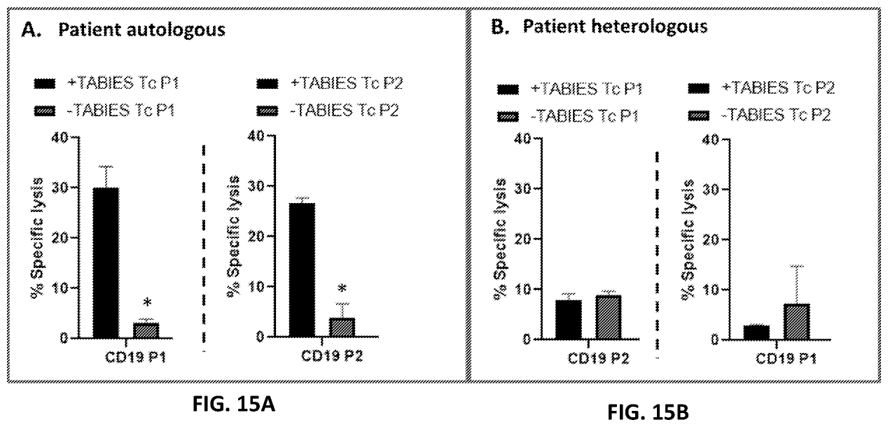

FIGS. 15A and 15B shows that Tumor Apoptotic Body Immunizations (TABI) stimulate patient tumor-specific cytolytic T-cell activity: CD19+ CLL cells were isolated from the PBMCs of CLL patient 1 (P1) and CLL patient 2 (P2) and were then split into 2 aliquots/patient sample: Aliquot 1 was used to generate TABI's by exposing CD19+ cells from each patient to daratumumab (30 μM, 18h); followed by isolation of TABI's using low speed centrifugation. From Aliquot 2: CD19+ CLL cells were labeled with Calcein-AM (5 μM/30 min in dark at 37° C.) and are shown as CD19 P1 or P2. Separately, from each patients PBMCs, CD8+ T-cells (Tc) were isolated and incubated with patient-autologous TABI's (+ TABis Tc P1 or P2) or PBS for 72 hr. FIG. 15A. After washing excess calcein AM, CD19+ cells from P1 or P2 were co-cultured with corresponding autologous cTL that were pre-exposed to patient-autologous TABI (i.e. CD19 P1 with Tc P1). FIG. 15B. In parallel, a patient-heterologous experiment was performed where calcein AM labeled CD19 P1 cells were cocultured with TABI-exposed cTL from P2 (Tc P2) and CD19 P2 cells were cocultured with TABI-exposed cTL from P2 (Tc P1). Calcein fluorescence was measured as in panel A. Data shown are representative of at least three independent experiments. *p<0.05

For flow cytometric analysis, CD19+ sorted CLL cells were stained with a combination of Hoechst 33342/Annexin V-FITC/TO-PRO-3 (DNA content) MitoTracker Green/Annexin V-V450/TO-PRO-3 (mitochondrial content), Hoechst 33342/MitoTracker Green/AnnexinV-APC (DNA and mitochondrial content), or Hoechst 33342/SYTO RNAselect green/Annexin V-APC (DNA and RNA content). Cells were stained with Hoechst 33342 (5 μg/ml), MitoTracker Green (100 nM) and/or SYTO RNAselect green (500 nM) prior to induction of apoptosis. Cells were stained with Annexin V and/or TO-PRO-3 in Annexin V binding buffer (1×) for 10 min at room temperature and immediately placed on ice before analysis on an Attune NxT flow cytometer (invitrogen). Unstained samples were used as a control. All data were analyzed using FCS Express 6 (DeNovo Software). Distribution of intracellular content of TABis varies with the mechanism of apoptotic cell disassembly using the various drugs. The distribution of DNA and mitochondria into TABis quantified based on (FIG. 13) TABis DNA distribution index and TABis mitochondria distribution index (FIG. 13) (n=3). Data are representative of at least three independent experiments. DNA distribution index=DNA+ TABis/DNA− TABis; Mitochondria distribution index=mitochondria+ TABis/mitochondria− TABis.

TABLE 6
TAB-Stor comparison.

| | Approach: 1 | Approach: 2 | Approach: 3 |
|---|---|---|---|
| | TABi10 | TABi7.5 | TABStor |
| | (90% EV Free | (92.5% EV free | HEPES (12 mM), |
| | FBS + DMSO) | FBS + DMSO) | NaCl (130 mM), |
| | | | KCl (2. mM), |
| | | | MgCl (0.35 mM) |
| | | | pH 7.4 |
| | | | Added vesicle free |
| | | | autologous serum + |
| | | | Trehalose (1:1) |
| 4 degree | — | — | — |
| −80 degree | — | — | *+USED |
| Snap freeze | — | — | *+USED |

Thus, it was found that the isolation of TABis through a low-speed centrifugation yielded a high quality immunogenic TABis that was optimized for use in the same patient, and in amounts sufficient to provide multiple immunizations. Further, a novel storage medium was developed that yields a high yield immunization that lost little, if any, of its potency when compared to freshly isolated TABis. Finally, the TABis made, isolated and/or stored using the present invention enhanced patient-specific tumor cell lysis via CD8⁺ T-cells, when compared to unexposed CD8⁺ T-cells.

In one embodiment, the present invention includes a method of preparing apoptotic bodies from a tumor biopsy, comprising, consisting essentially of, or consisting of: obtaining human tumor cells from a subject; inducing apoptosis of the human tumor cells with a drug or a physical treatment; and collecting apoptotic bodies from the apoptotic human tumor cells by: centrifuging at a low speed to form a pellet and a low speed centrifugation supernatant; collecting the low speed centrifugation supernatant; and centrifuging the low speed centrifugation supernatant at a high speed to form a high speed centrifugation pellet and a high speed centrifugation supernatant; and isolating the high speed centrifugation pellet, wherein the immunogenic composition comprises drug-treated immunogenic Tumor Apoptotic Bodies (TABi) having a purity of at least 82 to 100 percent, and wherein the TABi are obtained in less than 2 hours. In one aspect, the step of centrifuging at low-speed is repeated prior to the high-speed centrifugation step. In another aspect, the step of inducing apoptosis is with more than one drug directed to that human tumor cell type. In another aspect, the method further comprises the step of obtaining the human tumor cells selecting the drug or physical treatment(s) to make subject-specific TABis, measuring the subject-specific for cTL proliferation in vitro, and then providing the subject-specific TABis depending on the cTL proliferation measured. In another aspect, the step of centrifuging at low-speed is at less than 100 g, or between 25 and 75 g. In another aspect, the step of centrifuging at high-speed is at greater than 1,500 g, or between 1,750 and 10,000 g. In another aspect, the method does not include a medium-speed centrifugation step, wherein the medium-speed centrifugation is between 100 and 1,500 g. In another aspect, the human tumor cells are from a liquid or a solid tumor. In another aspect, the human tumor cells are obtained from lung, breast, cervical, ovarian, esophageal, colon, rectum, neural, glioma, prostate, bladder, bone, pancreas, liver, ovary, testis, uterus, placenta, brain, cartilage, smooth muscle, striated muscle, fibrous tissue, blood vessel, lymph vessel, lymph node, adipose tissue, brain, kidney, pituitary gland, parathyroid, thyroid, bronchial, adrenal, stomach, large intestine, small intestine, skin, adenomas, sarcomas, carcinomas, leukemias, lymphomas, multiple myeloma or blastomas, or a combination of any thereof. In another aspect, the TABi are ToPro-3 and Annexin-V positive, and following the isolation step comprise greater than 82% of total cells. In another aspect, the TABi are autologous, allogeneic, or xenogeneic. In another aspect, the drug used to treat the tumor cells is selected from an antimetabolite agent, alkylating or alkylating-like agent, intercalating agent, topoisomerase I or II inhibitor, antimitotic agent, kinase inhibitor, proteasome inhibitor, Bcl-2 inhibitors, chemotherapy agents, monoclonal antibodies, biologics, targeted therapeutics, such as, butyrate derivatives, staurosporine, sulindac derivatives, inflammatory cytokines, 5-fluorouracil, capecitabine, gemcitabine, pemetrexed, methotrexate, pemetrexed, methotrexate, edatrexate, hydroxyurea, fludarabine, mercaptopurine, nitrogen mustards (mechlorethamine, cyclophosphamide, ifosfamide, melphalan and chlorambucil), aziridines (thiotepa), nitrosoureas (carmustine, lomustine, semustine), triazenes (dacarbazine and temozolomide) and platinum derivatives (cisplatin, oxaliplatin, carboplatin and satraplatin), doxorubicin, epirubicin, idarubicin, nemorubicin, mitoxantrone, etoposide, teniposide, paclitaxel, docetaxel, ixabepilone, vinblastine, vincristine, vindesine and vinorelbine, sorafenib, dasatinib, gefitinib, erlotinib, sunitinib, imatinib, nilotinib lapatinib, ibrutinib, acalabrutinib, bevacizumab (antibody to vascular endothelial growth factor), cetuximab, panitumumab, matuzumab, nimotuzumab (antibodies to epidermal growth factor receptor), trastuzumab, pertuzumab (antibodies to ErbB2), daratumumab (antibody to CD38), anti-CD20 inhibitors (rituximab, obinutuzumab, ofatumumab), topotecan, SN-38, CPT11, 9-nitrocamptothecin, or antineoplasic nucleoside analogs. In another aspect, the physical treatment for inducing apoptosis is by a physical method selected from: ionization, γ-irradiation, UV irradiation, heat shock, stress, serum deprivation, or a combination thereof. In another aspect, the TABi or the TABI are lyophilized. In another aspect, the TABi or TABI are cryogenically stored in a composition comprising HEPES, NaCl, KCl, MgCl, vesicle free autologous serum and Trehalose, adjusted to pH 7.4.

In one embodiment, the present invention includes an immunogenic composition made by a method comprising, consisting essentially of, or consisting of: obtaining human tumor cells from a subject; inducing apoptosis of the human tumor cells with a drug or a physical treatment; and collecting apoptotic bodies from the apoptotic human tumor cells by: centrifuging at a low-speed to form a pellet and a low speed centrifugation supernatant; collecting the low-speed centrifugation supernatant; and centrifuging the low speed centrifugation supernatant at a high speed to form a high-speed centrifugation pellet and a high-speed centrifugation supernatant; and isolating a pellet after the high-speed centrifugation, wherein the pellet comprises immunogenic Tumor Apoptotic Bodies (TABi) having a purity of at least 82 percent, and wherein the TABI are obtained in less than 2 hours. In one aspect, the step of centrifuging at low-speed is repeated prior to the high-speed centrifugation step. In another aspect, the step of inducing apoptosis is with more than one drug directed to that human tumor cell type. In another aspect, the step of centrifuging at low-speed is at less than 100 g, or between 25 and 75 g. In another aspect, the step of centrifuging at high-speed is at greater than 1,500 g, or between 1,750 and 10,000 g. In another aspect, the method does not include a medium-speed centrifugation step, wherein the medium-speed centrifugation is between 100 and 1,500 g. In another aspect, the human tumor cells are from a liquid or a solid tumor. In another aspect, the human tumor cells are obtained from lung, breast, cervical, ovarian, esophageal, colon, rectum, neural, glioma, prostate, bladder, bone, pancreas, liver, ovary, testis, uterus, placenta, brain, cartilage, smooth muscle, striated muscle, fibrous tissue, blood vessel, lymph vessel, lymph node, adipose tissue, brain, kidney, pituitary gland, parathyroid, thyroid, bronchial, adrenal, stomach, large intestine, small intestine, skin, adenomas, sarcomas, carcinomas, leukemias, lymphomas, multiple myeloma or blastomas, or a combination of any thereof. In another aspect, the TABi or TABI are ToPro-3 and Annexin-V positive, and following the isolation step comprise greater than 82% of total cells. In another aspect, the TABi or TABI are autologous, allogeneic, or xenogeneic. In another aspect, the used to treat the tumor cells is selected from an antimetabolite agent, alkylating or alkylating-like agent, intercalating agent, topoisomerase I or II inhibitor, antimitotic agent, kinase inhibitor, proteasome inhibitor, Bcl-2 inhibitors, chemotherapy agents, monoclonal antibodies, biologics, targeted therapeutics, such as, butyrate derivatives, staurosporine, sulindac derivatives, inflammatory cytokines, 5-fluorouracil, capecitabine, gemcitabine, pemetrexed, methotrexate, pemetrexed, methotrexate, edatrexate, hydroxyurea, fludarabine, mercaptopurine, nitrogen mustards (mechlorethamine, cyclophosphamide, ifosfamide, melphalan and chlorambucil), aziridines (thiotepa), nitrosoureas (carmustine, lomustine, semustine), triazenes (dacarbazine and temozolomide) and platinum derivatives (cisplatin, oxaliplatin, carboplatin and satraplatin), doxorubicin, epirubicin, idarubicin, nemorubicin, mitoxantrone, etoposide, teniposide, paclitaxel, docetaxel, ixabepilone, vinblastine, vincristine, vindesine and vinorelbine, sorafenib, dasatinib, gefitinib, erlotinib, sunitinib, imatinib, nilotinib lapatinib, ibrutinib, acalabrutinib, bevacizumab (antibody to vascular endothelial growth factor), cetuximab, panitumumab, matuzumab, nimotuzumab (antibodies to epidermal growth factor receptor), trastuzumab, pertuzumab (antibodies to ErbB2), daratumumab (antibody to CD38), anti-CD20 inhibitors (rituximab, obinutuzumab, ofatumumab), topotecan, SN-38, CPT11, 9-nitrocamptothecin, or antineoplasic nucleoside analogs. In another aspect, the physical treatment for inducing apoptosis is by a physical method selected from: ionization, γ-irradiation, UV irradiation, heat shock, stress, serum deprivation, or a combination thereof. In another aspect, the TABi or TABI are lyophilized. In another aspect, the TABi are cryogenically stored in a composition comprising HEPES, NaCl, KCl, MgCl, vesicle free autologous serum and Trehalose, adjusted to pH 7.4. In another aspect, the method further comprises the step of obtaining the human tumor cells selecting the drug or physical treatment(s) to make subject-specific TABis, measuring the subject-specific for cTL proliferation in vitro, and then providing the subject-specific TABIs depending on the cTL proliferation measured.

A method of preparing apoptotic bodies from a tumor biopsy, comprising, consisting essentially of, or consisting of: obtaining human tumor cells from a subject; inducing apoptosis of the human tumor cells with a drug or a physical treatment; and collecting apoptotic bodies from the apoptotic human tumor cells by: centrifuging at a low speed of less than or equal to 100 g to form a pellet and a low speed centrifugation supernatant; collecting the low speed centrifugation supernatant; and centrifuging the low speed centrifugation supernatant at a high speed of greater than 1,500 g to form a high speed centrifugation pellet and a high speed centrifugation supernatant; and isolating the high speed centrifugation pellet, wherein the immunogenic composition comprises drug-treated immunogenic Tumor Apoptotic Bodies (TABi) having a purity of at least 82 to 100 percent, and wherein the TABi are obtained in less than 2 hours.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Coulie, P. G., Van den Eynde, B. J., van der Bruggen, P. & Boon, T. Tumour antigens recognized by T lymphocytes: at the core of cancer immunotherapy. Nat. Rev. Cancer 14, 135-146 (2014).
Bachireddy, P., Burkhardt, U. E., Rajasagi, M. & Wu, C. J. Haematological malignancies: at the forefront of immunotherapeutic innovation. Nat. Rev. Cancer 15, 201-215 (2015).
Restifo, N. P., Dudley, M. E. & Rosenberg, S. A. Adoptive immunotherapy for cancer: harnessing the T cell response. Nat. Rev. Immunol. 12, 269-281 (2012).
Burkhardt, U. E. et al. Autologous CLL cell vaccination early after transplant induces leukemia-specific T cells. J. Clin. Invest. 123, 3756-3765 (2013).
Kokhaei Pl, Choudhury A, Mandian R, Lundin J, Moshfegh A, Osterborg A, Mellstedt H. Apoptotic tumor cells are superior to tumor cell lysate, and tumor cell RNA in induction of autologous T cell response in B-CLL. Leukemia. 2004 November; 18(11):1810-5.
Keenan B P, Jaffee E M. Whole cell vaccines—past progress and future strategies. Semin Oncol. 2012 June; 39(3):276-86.
Thomas A, Santarsiero L, Lutz E, Armstrong T, Chen Y, Huang L, et al. Mesothelin-specific CD8(+) T cell responses provide evidence of in vivo cross-priming by antigen-presenting cells in vaccinated pancreatic cancer patients. J Exp Med. 2004; 200:297-306.
Shirota H, Klinman D M. CpG-conjugated apoptotic tumor cells elicit potent tumor-specific immunity. Cancer Immunol Immunother. 2011; 60:659-69.

1. A method of preparing apoptotic bodies from a tumor biopsy, comprising:
obtaining human tumor cells from a subject;
inducing apoptosis of the human tumor cells with a drug or a physical treatment;
collecting apoptotic bodies from the apoptotic human tumor cells by:
centrifuging at a low speed to form a pellet and a low speed centrifugation supernatant;
collecting the low speed centrifugation supernatant; and
centrifuging the low speed centrifugation supernatant at a high speed to form a high speed centrifugation pellet and a high speed centrifugation supernatant; and
isolating the high speed centrifugation pellet, wherein an immunogenic composition comprises drug-treated immunogenic Tumor Apoptotic Bodies (TABi) having a purity of at least 82 to 100 percent, and wherein the TABi are obtained in less than 2 hours.

2. (canceled)

3. The method of claim 1, wherein the step of inducing apoptosis is with more than one drug or physical treatment directed to that human tumor cell type.

4. The method of claim 3, further comprising the step of obtaining the human tumor cells, selecting the drug or physical treatment(s) to make subject-specific TABis, measuring cTL proliferation in vitro to the subject-specific TABis, and then providing the subject-specific TABis to the subject depending on the cTL proliferation measured.

5. The method of claim 1, wherein at least one of:
the step of centrifuging at low-speed is at less than 100 g, or between 25 and 75 g;
the step of centrifuging at low-speed is repeated prior to the high-speed centrifugation step;
the step of centrifuging at high-speed is at greater than 1,500 g, or between 1,750 and 10,000 g; or
the method does not include a medium-speed centrifugation step, wherein the medium-speed centrifugation is between 100 and 1,500 g.

6. (canceled)

7. (canceled)

8. The method of claim 1, wherein the human tumor cells are from a liquid or a solid tumor or the human tumor cells are obtained from lung, breast, cervical, ovarian, esophageal, colon, rectum, neural, glioma, prostate, bladder, bone, pancreas, liver, ovary, testis, uterus, placenta, brain, cartilage, smooth muscle, striated muscle, fibrous tissue, blood vessel, lymph vessel, lymph node, adipose tissue, kidney, pituitary gland, parathyroid, thyroid, bronchial, adrenal, stomach, large intestine, small intestine, skin, adenomas, sarcomas, carcinomas, leukemias, lymphomas, multiple myeloma or blastomas, or a combination of any thereof.

9. (canceled)

10. The method of claim 1, wherein the TABi are ToPro-3 and Annexin-V positive, and following the isolation step comprise greater than 82% of total cells or the TABi are autologous, allogeneic, or xenogeneic.

11. (canceled)

12. The method of claim 1, wherein the drug used to treat the human tumor cells is selected from an antimetabolite agent, alkylating or alkylating-like agent, intercalating agent, topoisomerase I or II inhibitor, antimitotic agent, kinase inhibitor, proteasome inhibitor, Bcl-2 inhibitors, chemotherapy agents, monoclonal antibodies, biologics, targeted therapeutics, butyrate derivatives, staurosporine, sulindac derivatives, inflammatory cytokines, 5-fluorouracil, capecitabine, gemcitabine, pemetrexed, methotrexate, pemetrexed, methotrexate, edatrexate, hydroxyurea, fludarabine, mercaptopurine, nitrogen mustards (mechlorethamine, cyclophosphamide, ifosfamide, melphalan and chlorambucil), aziridines (thiotepa), nitrosoureas (carmustine, lomustine, semustine), triazenes (dacarbazine and temozolomide) and platinum derivatives (cisplatin, oxaliplatin, carboplatin and satraplatin), doxorubicin, epirubicin, idarubicin, nemorubicin, mitoxantrone, etoposide, teniposide, paclitaxel, docetaxel, ixabepilone, vinblastine, vincristine, vindesine and vinorelbine, sorafenib, dasatinib, gefitinib, erlotinib, sunitinib, imatinib, nilotinib lapatinib, ibrutinib, acalabrutinib, bevacizumab (antibody to vascular endothelial growth factor), cetuximab, panitumumab, matuzumab, nimotuzumab (antibodies to epidermal growth factor receptor), trastuzumab, pertuzumab (antibodies to ErbB2), daratumumab (antibody to CD38), anti-CD20 inhibitors (rituximab, obinutuzumab, ofatumumab), topotecan, SN-38, CPT11, 9-nitrocamptothecin, or antineoplasic nucleoside analogs.

13. The method of claim 1, wherein the physical treatment for inducing apoptosis is by a physical method selected from: ionization, γ-irradiation, UV irradiation, heat shock, stress, serum deprivation, or a combination thereof.

14. The method of claim 1, wherein the TABi are lyophilized or the TABi are cryogenically stored in an aqueous solution comprising HEPES, NaCl, KCl, MgCl, vesicle free autologous serum and Trehalose, adjusted to pH 7.4.

15. (canceled)

16. An immunogenic Tumor Apoptotic Body Immunization (TABI) composition made by a method comprising:
obtaining human tumor cells from a subject;
inducing apoptosis of the human tumor cells with a drug or a physical treatment;
collecting apoptotic bodies from the apoptotic human tumor cells by:
centrifuging at a low-speed to form a pellet and a low speed centrifugation supernatant;
collecting the low-speed centrifugation supernatant; and
centrifuging the low speed centrifugation supernatant at a high speed to form a high-speed centrifugation pellet and a high-speed centrifugation supernatant; and
isolating the high-speed centrifugation pellet, wherein the pellet comprises immunogenic Tumor Apoptotic Bodies (TABi) having a purity of at least 82 percent, and wherein the TABi are obtained in less than 2 hours.

17. (canceled)

18. The composition of claim 16, wherein the step of inducing apoptosis is with more than one drug directed to that human tumor cell type.

19. The composition of claim 16, at least one of:
the step of centrifuging at low-speed is at less than 100 g, or between 25 and 75 g;
the step of centrifuging at low-speed is repeated prior to the high-speed centrifugation step;
the step of centrifuging at high-speed is at greater than 1,500 g, or between 1,750 and 10,000 g; or the method does not include a medium-speed centrifugation step, wherein the medium-speed centrifugation is between 100 and 1,500 g.

20. (canceled)

21. (canceled)

22. The composition of claim 16, wherein the human tumor cells are from a liquid or a solid tumor or the human tumor cells are obtained from lung, breast, cervical, ovarian, esophageal, colon, rectum, neural, glioma, prostate, bladder, bone, pancreas, liver, ovary, testis, uterus, placenta, brain, cartilage, smooth muscle, striated muscle, fibrous tissue, blood vessel, lymph vessel, lymph node, adipose tissue, kidney, pituitary gland, parathyroid, thyroid, bronchial, adrenal, stomach, large intestine, small intestine, skin, adenomas, sarcomas, carcinomas, leukemias, lymphomas, multiple myeloma or blastomas, or a combination of any thereof.

23. (canceled)

24. The composition of claim 16, wherein the TABi are ToPro-3 and Annexin-V positive, and following the isolation step comprise greater than 82% of total cells or the TABi are autologous, allogeneic, or xenogeneic.

25. (canceled)

26. The composition of claim 16, wherein the drug used to treat the human tumor cells is selected from an antimetabolite agent, alkylating or alkylating-like agent, intercalating agent, topoisomerase I or II inhibitor, antimitotic agent, kinase inhibitor, proteasome inhibitor, Bcl-2 inhibitors, chemotherapy agents, monoclonal antibodies, biologics, targeted therapeutics, butyrate derivatives, staurosporine, sulindac derivatives, inflammatory cytokines, 5-fluorouracil, capecitabine, gemcitabine, pemetrexed, methotrexate, pemetrexed, methotrexate, edatrexate, hydroxyurea, fludarabine, mercaptopurine, nitrogen mustards (mechlorethamine, cyclophosphamide, ifosfamide, melphalan and chlorambucil), aziridines (thiotepa), nitrosoureas (carmustine, lomustine, semustine), triazenes (dacarbazine and temozolomide) and platinum derivatives (cisplatin, oxaliplatin, carboplatin and satraplatin), doxorubicin, epirubicin, idarubicin, nemorubicin, mitoxantrone, etoposide, teniposide, paclitaxel, docetaxel, ixabepilone, vinblastine, vincristine, vindesine and vinorelbine, sorafenib, dasatinib, gefitinib, erlotinib, sunitinib, imatinib, nilotinib lapatinib, ibrutinib, acalabrutinib, bevacizumab (antibody to vascular endothelial growth factor), cetuximab, panitumumab, matuzumab, nimotuzumab (antibodies to epidermal growth factor receptor), trastuzumab, pertuzumab (antibodies to ErbB2), daratumumab (antibody to CD38), anti-CD20 inhibitors (rituximab, obinutuzumab, ofatumumab), topotecan, SN-38, CPT11, 9-nitrocamptothecin, or antineoplasic nucleoside analogs.

27. The composition of claim 16, wherein the physical treatment for inducing apoptosis is by a physical method selected from: ionization, γ-irradiation, UV irradiation, heat shock, stress, serum deprivation, or a combination thereof.

28. The composition of claim 16, wherein the TABi or the TABI are lyophilized or the TABi are cryogenically stored in an aqueous solution comprising HEPES, NaCl, KCl, MgCl, vesicle free autologous serum and Trehalose, adjusted to pH 7.4.

29. (canceled)

30. The composition of claim 16, further comprising the step of obtaining the human tumor cells selecting the drug or physical treatment(s) to make subject-specific TABIs, measuring cTL proliferation in vitro to the subject-specific TABIs, and then providing the subject-specific TABIs to the subject depending on the cTL proliferation measured.

31. A method of preparing apoptotic bodies from a tumor biopsy, comprising:
obtaining human tumor cells from a subject;
inducing apoptosis of the human tumor cells with a drug or a physical treatment; and
collecting apoptotic bodies from the apoptotic human tumor cells by:
centrifuging at a low speed of less than or equal to 100 g to form a pellet and a low speed centrifugation supernatant;
collecting the low speed centrifugation supernatant; and
centrifuging the low speed centrifugation supernatant at a high speed of greater than 1,500 g to form a high speed centrifugation pellet and a high speed centrifugation supernatant; and
isolating the high speed centrifugation pellet, wherein an immunogenic composition comprises drug-treated immunogenic Tumor Apoptotic Bodies (TABi) having a purity of at least 82 to 100 percent, and wherein the TABI are obtained in less than 2 hours.
